(19) United States
(12) Patent Application Publication
(10) Pub. No.: US 2013/0211198 A1
(43) Pub. Date: Aug. 15, 2013

(54) ENDOSCOPIC DEVICE

(71) Applicant: FUJIFILM CORPORATION, (US)

(72) Inventors: Nobuyuki TORISAWA, Ashigarakami-gun (JP); Masayuki IWASAKA, Ashigarakami-gun (JP)

(73) Assignee: FUJIFILM CORPORATION, Tokyo (JP)

(21) Appl. No.: 13/754,207

(22) Filed: Jan. 30, 2013

(30) Foreign Application Priority Data
Feb. 15, 2012 (JP) 2012-030169

(51) Int. Cl. A61B 1/12

(52) U.S. Cl. CPC A61B 1/126
USPC 600/157

(57) ABSTRACT

In order to prevent floating matters from attaching to a glass plate fixed on a leading end face of an endoscope insertion portion, gas-feed ports are formed around the glass plate. Between the glass plate and the gas-feed ports, a protective barrier is formed in a manner surrounding the glass plate. Ejection of gas through the gas-feed ports generates gas streams to remove unnecessary floating matters existing between the glass plate and an observation target from sight. The gas streams also generate secondary gas streams flowing toward the glass plate. Some of the secondary gas streams are drawn forward with the primary gas streams, while the other of the secondary gas streams collide with the protective barrier. The secondary gas streams cannot flow along the surface of the glass plate, which prevents floating matters flowing with the secondary gas streams from attaching to the surface of the glass plate.

### BACKGROUND OF THE INVENTION

1. Field of the Invention

The present invention relates to an endoscopic device to be inserted and take an image in a body.

2. Description of the Related Art

Rigid endoscopic devices are configured to take an image in a living body by inserting a rigid endoscope in the living body. An imaging lens is provided on the leading end face of the rigid endoscope and an image of a target is formed through the imaging lens on an imaging device built in the leading end portion of the rigid endoscope. Since the rigid endoscope is inserted in the living body, blood and/or mucus may attach to the imaging lens to cause visibility degradation. To address this problem, there has been proposed a technique of cleaning an imaging lens (see Japanese Unexamined Patent Application Publication No. Hei 5-199979, for example).

Further, during rigid-endoscopic treatment using an electrocautery, smoke generation may cause visibility degradation. There has hence been proposed a technique of ejecting harmless inert gas forward from the leading end face of a rigid endoscope (see Japanese Patent No. 3310349, for example). FIG. 22 is a front view of the leading end face of such a rigid endoscope insertion portion 200. A glass plate (lens) 204 provided on an observation port is attached to the leading end face of the rigid endoscope insertion portion 200 and, around the glass plate 204, ejection ports 201, 202, and 203 are formed for ejecting gas forward therethrough.

However, the technique described in Japanese Unexamined Patent Application Publication No. Hei 5-199979 do not take into account how to deal with visibility degradation of the imaging device due to floating of oil droplets, steam, and other matters between an observation target and the imaging lens, which may be caused by burning off the target site using an endoscope.

In the technique described in Japanese Patent No. 3310349, ejection of gas through the ejection ports 201, 202, and 203, if occurs (in the direction perpendicular to the paper surface), induces gas streams “wi” and “wo” as indicated by the arrows in FIG. 22. Some gas streams “wi” are drawn by the gas ejected through the ejection ports 201, 202, and 203 to flow forward from the rigid endoscope insertion portion 200 together with the gas, while the other gas streams “wo” may be drawn toward the surface of the glass plate 204. This may cause floating matters such as oil droplets to attach to the surface of the glass plate 204. Thus, in the technique described in Japanese Patent No. 3310349, ejection of gas through the ejection ports 201, 202, and 203 intended to improve the visibility of the imaging device may cause floating matters to attach to the surface of the glass plate 204, ending up further degrading the visibility.

### SUMMARY OF THE INVENTION

It is hence an object of the present invention to remove matters floating between an observation target and an observation optical member such as an imaging lens to ensure improved visibility and to prevent contaminants such as floating matters from attaching to the observation optical member.

The present invention is directed to an endoscopic device including: an endoscope insertion portion having a leading end portion, a base end portion, and a longitudinal shaft; an observation optical member (including a glass plate, an imaging lens, a plastic plate, a transparent plate, a translucent plate, and an optical member transparent to particular wavelengths) provided in the leading end portion of the endoscope insertion portion and having a surface exposed outward; a convex portion formed around the observation optical member; and a gas-feed port formed on the convex portion or on the outside of the convex portion with respect to the observation optical member and configured to eject gas therethrough in the direction of observation from the observation optical member, wherein said convex portion is more protrudent than the surface of the observation optical member in the direction of observation.

In accordance with the present invention, since the gas-feed port is formed for ejecting gas therethrough in the direction of observation from the observation optical member, floating matters such as oil droplets and steam, if may be generated when the endoscope insertion portion is inserted in a body and may cause visibility degradation, can be removed from sight. Further, since the convex portion is formed around the imaging window, ambient gas, if may be induced by gas ejected through the gas-feed port, cannot reach inside of the body or the imaging window, whereby it is possible to prevent oil droplets and the like contained in the induced ambient gas from attaching to the imaging window. It is therefore possible to remove matters floating between an observation target and the intravital observation optical member to ensure improved visibility and to prevent contaminants from attaching to the surface of the intravital observation optical member.

The convex portion is, for example, a protective barrier provided in a standing manner around the observation optical member.

It is preferable that at least one of the outer and inner walls of the protective barrier is inclined in a manner spreading downward.

The protective barrier may be provided between the observation optical member and the gas-feed port in a manner surrounding the observation optical member.

The protective barrier may be provided between the observation optical member and the gas-feed port only at a position facing the gas-feed port.

The protective barrier may not be provided between the observation optical member and the gas-feed port but may be provided at a position opposed to the gas-feed port with respect to the observation optical member.

The outer wall of the protective barrier may be inclined in a manner spreading downward.

The endoscopic device is, for example, a rigid endoscopic device or a flexible endoscopic device.

The endoscopic device may further include a gas-feed device for feeding the gas-feed port with gas to be ejected through the gas-feed port.

The gas-feed port may be formed all around the observation optical member.

The endoscopic device may further include an overtube for covering the endoscope insertion portion. In this case, multiple protrusions may be formed circumferentially at their respective different positions in the leading end portion of the overtube in a manner protruding inward, and the multiple protrusions formed on the overtube may be in contact with the outer peripheral surface of the endoscope insertion portion to provide a clearance gap all around the leading end face of the endoscope insertion portion between the endoscope insertion portion and the overtube serving as the gas-feed port.

The protrusions have, for example, a common height.

The endoscopic device may further include a deflection member for guiding part of gas ejected through the gas-feed port to the observation optical member.

The deflection member is, for example, provided in front of the gas-feed port and includes a support member provided in a manner standing in the longitudinal direction of the endoscope insertion portion and a bent member formed by bending an upper part of the support member toward the observation optical member.

### BRIEF DESCRIPTION OF THE DRAWINGS

FIG. 1 is a perspective view of an endoscope insertion portion.

FIG. 2 is a side view of the endoscope insertion portion.

FIG. 3 is a front view of the endoscope insertion portion.

FIG. 4 is a cross-sectional view taken along the line IV-IV of FIG. 3.

FIG. 5 shows an endoscopic device.

FIG. 6 is a perspective view of the endoscope insertion portion.

FIGS. 7 and 8 are front views of an endoscope insertion portion.

FIG. 9 is a cross-sectional view taken along the line IX-IX of FIG. 8.

FIG. 10 is a cross-sectional view of a leading end portion of an endoscope insertion portion.

FIG. 11 is a front view of an endoscope insertion portion.

FIG. 12 is a side view of the endoscope insertion portion.

FIG. 13 is a perspective view of an overtube.

FIG. 14 is a cross-sectional view taken along the line XIV-XIV of FIG. 13.

FIG. 15 is a perspective view of the overtube and an endoscope insertion portion.

FIG. 16 is a perspective view of the endoscope insertion portion covered with the overtube.

FIG. 17 is a cross-sectional view taken along the line XVII-XVII of FIG. 16.

FIG. 18 is a front view of the endoscope insertion portion covered with the overtube.

FIG. 19 is a front view of an endoscope insertion portion.

FIG. 20 is a side view of the endoscope insertion portion.

FIG. 21 is a perspective view of an endoscope insertion portion.

FIG. 22 is a front view of a conventional endoscope insertion portion.

### DESCRIPTION OF THE PREFERRED EMBODIMENTS

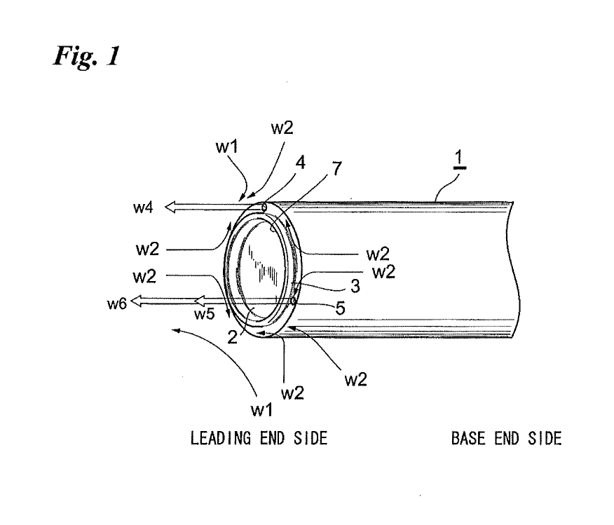

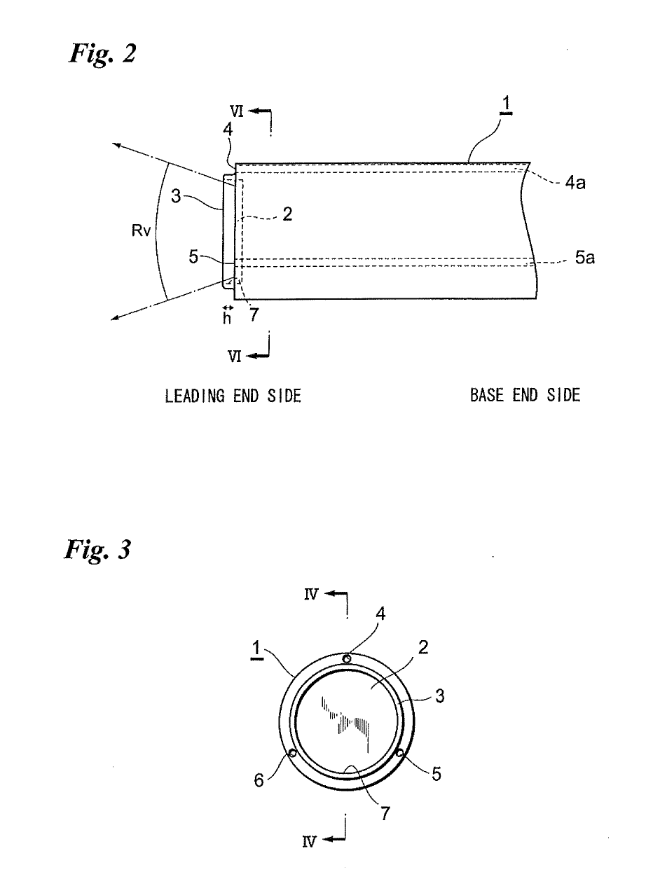

FIG. 1 is a perspective view of an endoscope insertion portion 1 of a rigid endoscope. FIG. 2 is a side view of the endoscope insertion portion 1. FIG. 3 is a front view of the endoscope insertion portion 1.

The endoscope insertion portion 1 has a longitudinal shaft with a circular leading end face on which an observation window 7 is formed for intravital observation. A circular glass plate (or a plastic plate or an imaging lens) 2 is attached to the observation window 7 for the purpose of, for example, preventing ingress of extraneous matters into the endoscope insertion portion 1. The intravital observation is made through the glass plate 2. A circular tubular protective barrier 3 is provided all around the circular glass plate 2. As shown in FIG. 2, the height “h” of the protective barrier 3 is determined not to block the intravital observer's sight Rv.

As shown in FIG. 3, three gas-feed ports 4, 5, and 6 configured to eject gas therethrough are formed on the outside of the protective barrier 3 with respect to the glass plate 2. Not three gas⁻feed ports 4, 5, and 6 but one or two or four or more gas-feed ports may be formed. The gas-feed ports 4, 5, and 6 may also be arranged circumferentially at equal or unequal intervals. As shown in FIG. 2, gas-feed pipes 4a and 5a through which gas flows run inside the endoscope insertion portion 1 and are connected to the respective gas-feed ports 4 and 5. Another gas-feed pipe is also connected to the gas-feed port 6, though not shown in FIG. 2. Gas is fed through these gas-feed pipes 4a and 5a and ejected through the gas-feed ports 4, 5, and 6.

Referring to FIG. 1, ejection of gas through the gas-feed ports 4, 5, and 6 in the direction of observation from the glass plate 2 of the endoscope insertion portion 1 generates gas streams w4, w5, and w6 as indicated by the arrows. These gas streams w4, w5, and w6 remove extraneous matters such as oil droplets from sight, if may exist in front of the glass plate 2. Ejection of gas through the gas-feed ports 4, 5, and 6 thus generates gas streams w4, w5, and w6, which in turn induces ambient gas around the gas streams w4, w5, and w6 to generate gas streams w1 and w2 as indicated by the arrows.

Among these gas streams w1 and w2, some gas streams w1 get caught in the gas streams w4, w5, and w6, which are generated by air ejected through the gas-feed ports 4, 5, and 6, to flow forward from the endoscope insertion portion 1. The other gas streams w2 do not get caught in the gas streams w4, w5, and w6, which are generated by air ejected through the gas-feed ports 4, 5, and 6, to partially flow toward the surface of the glass plate 2. In this preferred embodiment, the gas streams w2 collide with the protective barrier 3 and flow along the protective barrier 3 toward the gas-feed ports 4, 5, and 6. The gas streams w2 then get caught in the gas streams w4, w5, and w6, which are generated by air ejected through the gas-feed ports 4, 5, and 6, to flow forward from the endoscope insertion portion 1, as is the case with the gas streams w1. If the gas streams w2 may not partially flow along the protective barrier 3 toward the gas-feed ports 4, 5, and 6, the protective barrier 3 causes the gas streams w2 not to flow along the surface of the glass plate 2 but to flow forward from the endoscope insertion portion 1. In this preferred embodiment, the gas streams w1 and w2, if may be induced and generated by the gas streams w4, w5, and w6, which are generated by gas ejected through the gas-feed ports 4, 5, and 6, cannot flow along the surface of the glass plate 2. Even if extraneous matters may float with the gas streams w1 and w2, it is possible to prevent the extraneous matters from attaching to the glass plate 2.

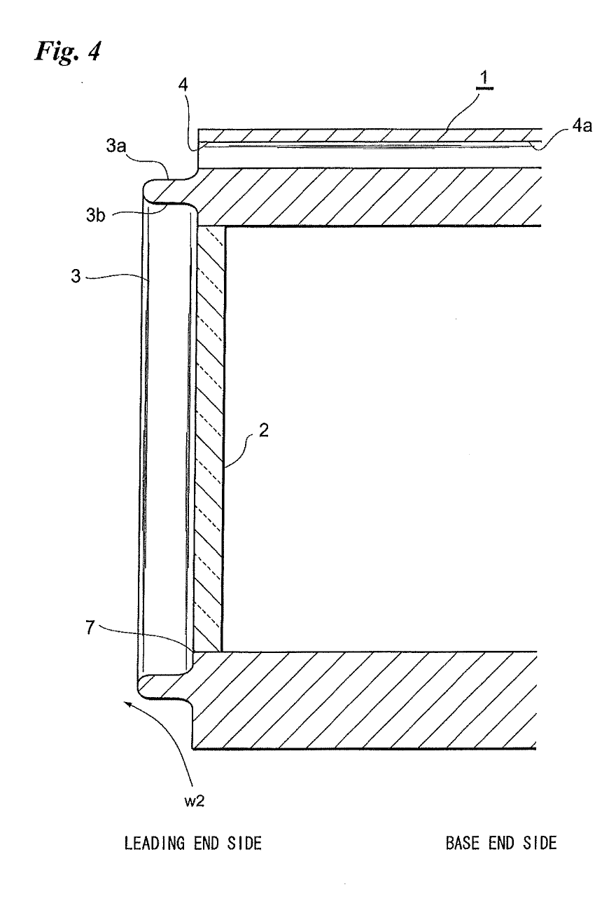

FIG. 4 is a partial cross-sectional view taken along the line IV-IV of FIG. 3. In FIG. 4, components identical to those described above are designated by the same reference numerals to omit the descriptions thereof.

The outer wall 3a and the inner wall 3b of the protective barrier 3 are both gently inclined in a manner spreading downward (from left to right in FIG. 4). Since the inner wall 3b is gently inclined, waterdrops and the like, if may be collected within the protective barrier 3, can be easily removed from within the protective barrier 3. Since the outer wall 3a is also gently inclined, gas streams flowing toward the glass plate 2 can easily flow along the outer wall 3a forward from the endoscope insertion portion 1.

Further, since the upper part of the protective barrier 3 is rounded, the protective barrier 3 cannot damage inside of the body. It will be understood that the upper part of the protective barrier 3 may not be rounded. It will also be understood that the outer and inner walls 3a and 3b may not be gently inclined but may be provided perpendicularly downward.

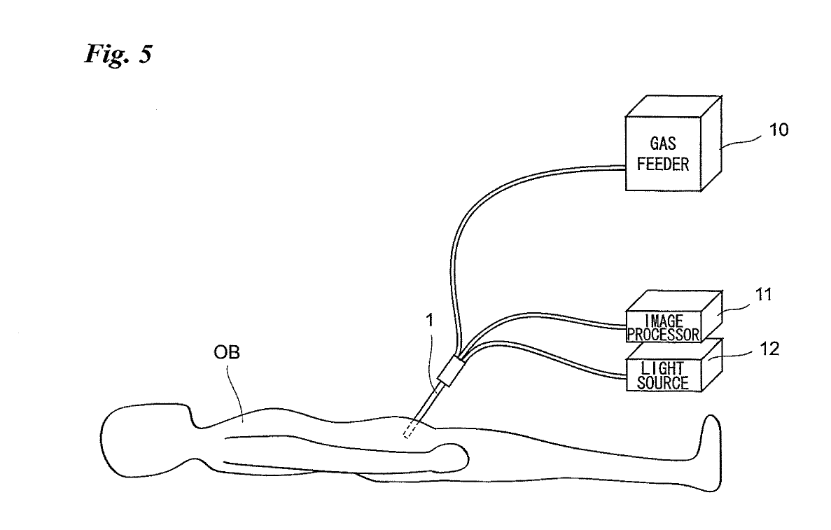

FIG. 5 schematically shows an endoscopic device according to a preferred embodiment of the present invention.

The endoscopic device includes the foregoing columnar endoscope insertion portion (endoscope) 1 to be inserted into the abdominal cavity of a subject OB. The endoscope insertion portion 1 is connected with a gas-feed device 10 for feeding air, an image processor 11, and a light source device 12 producing a light source for illuminating the body cavity.

The endoscopic device is configured to insert the endoscope insertion portion 1 into the abdominal cavity of the subject OB, take an image in the abdominal cavity of the subject OB, and display the taken image on a display screen. The operator then has surgery viewing the image on the display screen.

In this preferred embodiment, air fed from the gas-feed device 10 flows through the gas-feed pipes within the endoscope insertion portion 1 to be ejected through the gas-feed ports 4, 5, and 6 of the endoscope insertion portion 1. That is, the air is fed from the endoscope insertion portion 1 into the abdominal cavity of the subject OB.

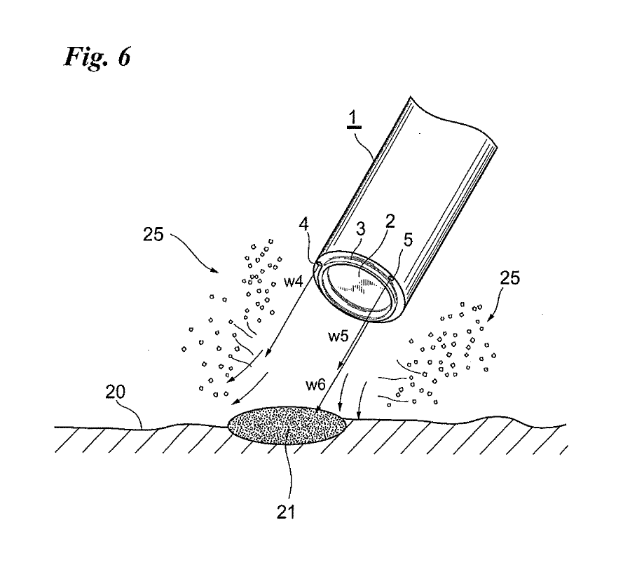

FIG. 6 shows the leading end portion of the endoscope insertion portion 1 inserted in the abdominal cavity.

An imaging device is built in the leading end portion of the endoscope insertion portion 1 and configured to take an image of an observation site 21 within the abdominal cavity 20 of the subject OB through the glass plate 2 on the leading end face 10 of the endoscope insertion portion 1 (light illumination window is not shown in this figure).

In this preferred embodiment, gas is ejected through the gas-feed ports 4 to 6 formed in the leading end face 10 of the endoscope insertion portion 1 forward in the longitudinal direction of the endoscope insertion portion 1 (i.e. in the direction of observation in which the observation site 21 exists or in the direction of imaging), as mentioned above, to generate gas streams w4 to w6. The gas streams w4 to w6 remove floating matters 25 such as oil droplets and steam if may float between the glass plate 2 and the observation site 21. This ensures improved visibility and improves the quality of the image of the observation site 21. Particularly in this preferred embodiment, the protective barrier 3 is formed between the glass plate 2 and the gas-feed ports 4, 5, and 6 in a manner surrounding the glass plate 2 as mentioned above, which prevents other gas streams w1 and w2 generated by the gas streams w4, w5, and w6 from flowing toward the surface of the glass plate 2 and thereby floating matters 25 contained in the gas streams w1 and w2 from attaching to the glass plate 2.

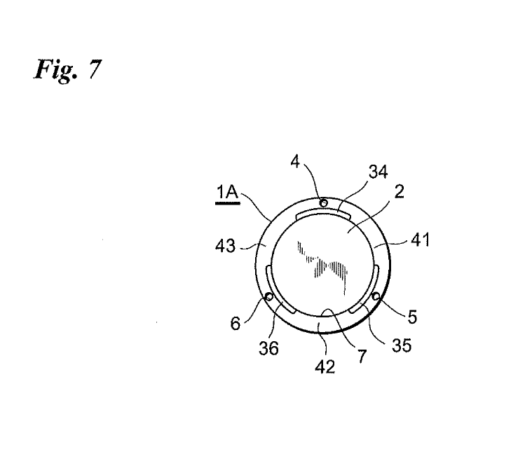

FIG. 7 is a front view showing a leading end face of an endoscope insertion portion 1A according to another preferred embodiment. FIG. 7 corresponds to FIG. 3.

Gas-feed ports 4, 5, and 6 are formed in the leading end face of the endoscope insertion portion 1A at equal circumferential intervals. First, second, and third protective barriers 34, 35, and 36 are provided in a standing manner, respectively, between the glass plate 2 attached to the observation window 7 and the gas-feed port 4, between the glass plate 2 and the gas-feed port 5, and between the glass plate 2 and the gas-feed port 6. The first, second, and third protective barriers 34, 35, and 36 have an arc shape in front view with the both ends thereof being gently rounded. The first, second, and third protective barriers 34, 35, and 36 are separated in the circumferential direction and no protective barrier is provided between the first, second, and third protective barriers 34, 35, and 36 (between the gas-feed ports 4, 5, and 6 in the circumferential direction). Also in the example shown in FIG. 7, the gas-feed ports 4, 5, and 6 are formed on the outside of the protective barriers 34, 35, and 36 with respect to the glass plate 2.

If gas streams w1 may be induced and generated by the gas streams w4, w5, and w6, which are generated by gas ejected through the gas-feed ports 4, 5, and 6 as mentioned above (see FIG. 1), the protective barriers 34, 35, and 36 can prevent the gas streams w1 from flowing toward the surface of the glass plate 2. Thus providing the protective barriers 34, 35, and 36 at positions corresponding to the gas-feed ports 4, 5, and 6 can prevent the gas streams from flowing toward the surface of the glass plate 2, even without forming protective barriers at positions corresponding to the gas-feed ports 4, 5, and 6.

The first, second, and third protective barriers 34, 35, and 36, though have an arc shape, may be linear as long as provided, respectively, between the glass plate 2 and the gas-feed ports 4, 5, and 6.

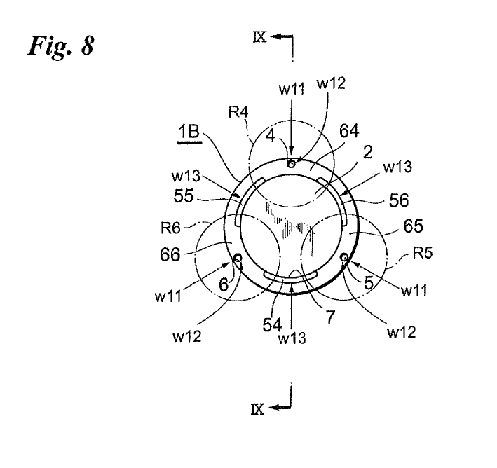

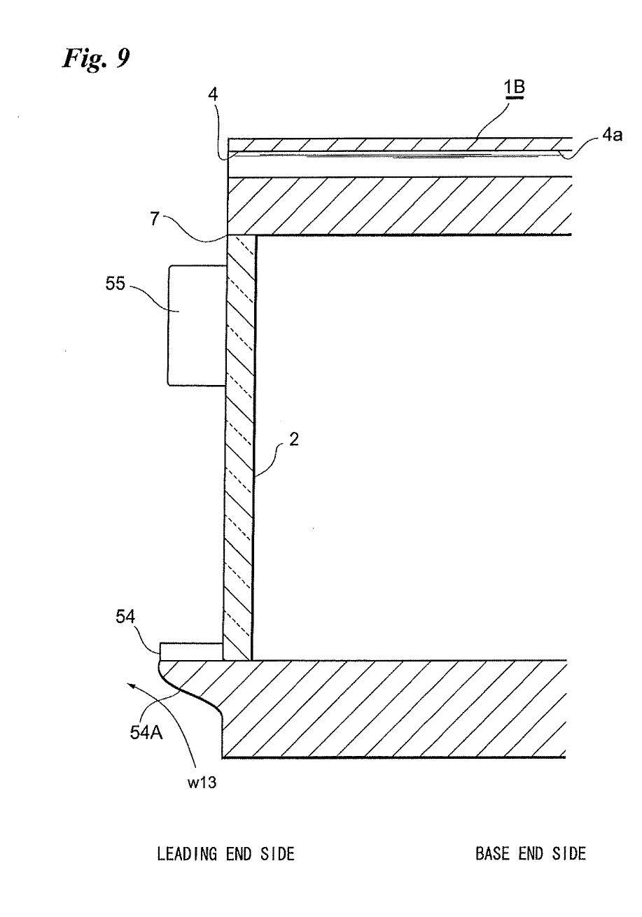

FIGS. 8 and 9 show still another preferred embodiment.

FIG. 8 is a front view showing a leading end face of an endoscope insertion portion 1B. FIG. 8 corresponds to FIGS. 3 and 7.

Unlike the endoscope insertion portion 1A shown in FIG. 7, the endoscope insertion portion 1B shown in FIG. 8 includes no protective barrier provided between the glass plate 2 attached to the observation window 7 and the gas-feed port 4, between the glass plate 2 and the gas-feed port 5, and between the glass plate 2 and the gas-feed port 6. Instead a first protective barrier 54 is provided at a position opposed to the gas-feed port 4 with respect to the glass plate 2, a second protective barrier 55 is provided at a position opposed to the gas-feed port 5 with respect to the glass plate 2, and a third protective barrier 56 is provided at a position opposed to the gas-feed port 6 with respect to the glass plate 2. Also in the example shown in FIG. 8, the gas-feed ports 4, 5, and 6 are formed on the outside of the protective barriers 54, 55, and 56 with respect to the glass plate 2. Specifically, the gas-feed ports 4, 5, and 6 are provided at positions farther from the center of the glass plate 2 than the protective barriers 54, 55, and 56.

A range R4 within which ambient gas is induced by the gas stream w4, which is generated by gas ejected through the gas-feed port 4, a range R5 within which ambient gas is induced by the gas stream w5, which is generated by gas ejected through the gas-feed port 5, and a range R6 within which ambient gas is induced by the gas stream w6, which is generated by gas ejected through the gas-feed port 6, are also shown. As mentioned above, ejection of gas through the gas-feed ports 4, 5, and 6 generates gas streams w4, w5, and w6 in front of the gas-feed ports 4, 5, and 6, which in turn generates gas streams w11, w12, and w13 as indicated by the arrows. Among these gas streams w11, w12, and w13, the gas streams w11 and w12, which enter the ranges R4, R5, and R6 formed around the gas-feed ports 4, 5, and 6 within which ambient gas is induced, are caught in the gas streams w4, w5, and w6, which are generated by air ejected through the respective gas-feed ports 4, 5, and 6, to flow forward from the endoscope insertion portion 1B. On the other hand, the gas stream w13, which does not enter the range R4, R5, or R6 within which ambient gas is induced but flows toward the surface of the glass plate 2, collides with one of the first, second, and third protective barriers 54, 55, and 56. The gas stream w13 thus cannot flow along the surface of the glass plate 2, which can prevent floating matters flowing with the gas stream w13 from attaching to the surface of the glass plate 2.

FIG. 9 is a cross-sectional view taken along the line IX-IX of FIG. 8. In FIG. 9, components identical to those shown in FIG. 8 are designated by the same reference numerals to omit the descriptions thereof.

The outer walls of the first, second, and third protective barriers 54, 55, and 56 (only the outer wall 54A of the first protective barrier 54 is shown in FIG. 9) are gently inclined in a manner spreading downward (from left to right in FIG. 9). This causes, for example, the gas stream w13 flowing toward the first protective barrier 54 to flow forward (leftward in FIG. 9) along the inclined outer wall 54A. This can prevent the gas stream w13 from flowing along the surface of the glass plate 2.

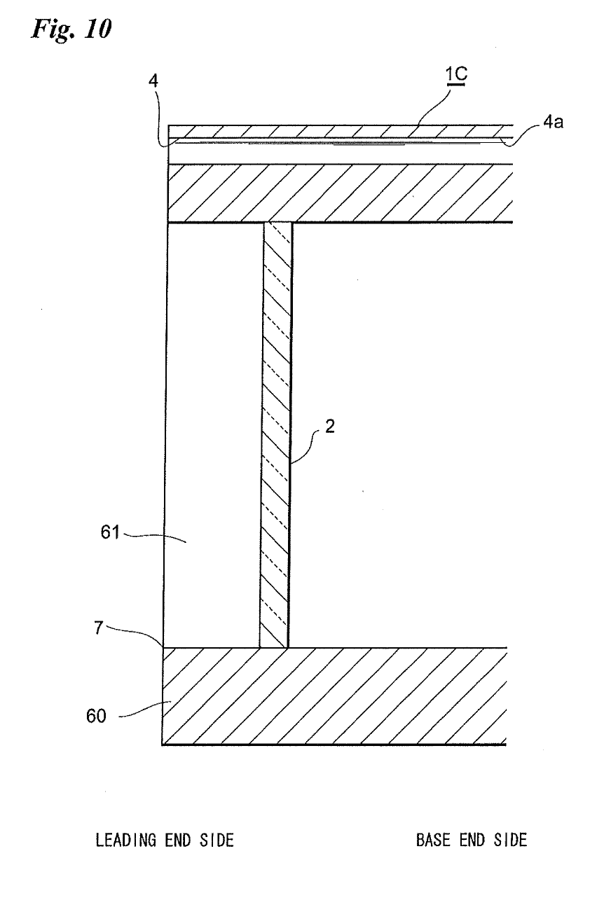

FIG. 10 is a side cross-sectional view of an endoscope insertion portion 1C according to a further preferred embodiment. FIG. 10 corresponds to FIGS. 4 and 9.

In this preferred embodiment shown in FIG. 10, the glass plate 2 is attached inside the observation window 7 nearer the base end than the endoscope leading end face 60, though attached to the leading end face of the endoscope insertion portion in the above-described endoscope insertion portion 1 and the like. As mentioned above, the glass plate 2 serves as a surface exposed outward and the leading end portion (convex portion) 61 of the endoscope insertion portion 1C protrudes from the glass plate 2. Thus attached nearer the base end than the endoscope leading end face 60, the glass plate 2 is surrounded by the leading end portion (convex portion) 61 of the endoscope insertion portion 1C, whereby the leading end portion 61 can prevent gas streams from flowing onto the surface of the glass plate 2 as mentioned above. Thus retracting the glass plate 2 from the leading end face 60 of the endoscope insertion portion 1C can also prevent matters floating with gas streams from attaching to the surface of the glass plate 2.

FIGS. 11 and 12 show a still further preferred embodiment.

FIG. 11 is a front view showing a leading end face of an endoscope insertion portion 1D, and FIG. 12 is a side view of the endoscope insertion portion 1D.

Referring to FIG. 11, a glass plate 2 is attached to the leading end face of the endoscope insertion portion 1D and a gas-feed port 80 is formed all around the glass plate 2.

Referring to FIG. 12, gas-feed pipes 92, 102, and 112 are formed in the longitudinal direction inside the endoscope insertion portion 1D (gas-feed pipes formed behind the gas-feed pipe 102 are not shown in FIG. 12). The gas-feed pipes 92, 102, and 112 have a fan shape spreading gradually toward the leading end. The fan-shaped portion 91 of the gas-feed pipe 92 and the fan-shaped portion 101 of the gas-feed pipe 102 merge in the vicinity of the leading end face of the endoscope insertion portion 1D (to form a fixed portion 71). Similarly, the fan-shaped portion 101 of the gas-feed pipe 102 and the fan-shaped portion 111 of the gas-feed pipe 112 merge in the vicinity of the leading end face of the endoscope insertion portion 1D (to form a fixed portion 72). The gas-feed pipes on the opposite side similarly have a fan shape spreading gradually toward the leading end and merge in the vicinity of the leading end face of the endoscope insertion portion 1D (to form fixed portions 73 and 74 shown in FIG. 11), though not shown in FIG. 12. The glass plate 2 is attached to the observation window 7 of the endoscope insertion portion 1D via the portions 71 to 74 where the fan-shaped portions merge.

Ejection of air through the gas-feed port 80 generates a gas stream surrounding the glass plate 2. This gas stream blocks gas streams flowing inward to the glass plate 2. The gas stream generated by air ejected through the gas-feed port 80 serves as a so-called air curtain to block gas streams flowing inward to the glass plate 2. This can prevent matters floating with the gas streams flowing inward to the glass plate 2 from attaching to the glass plate 2.

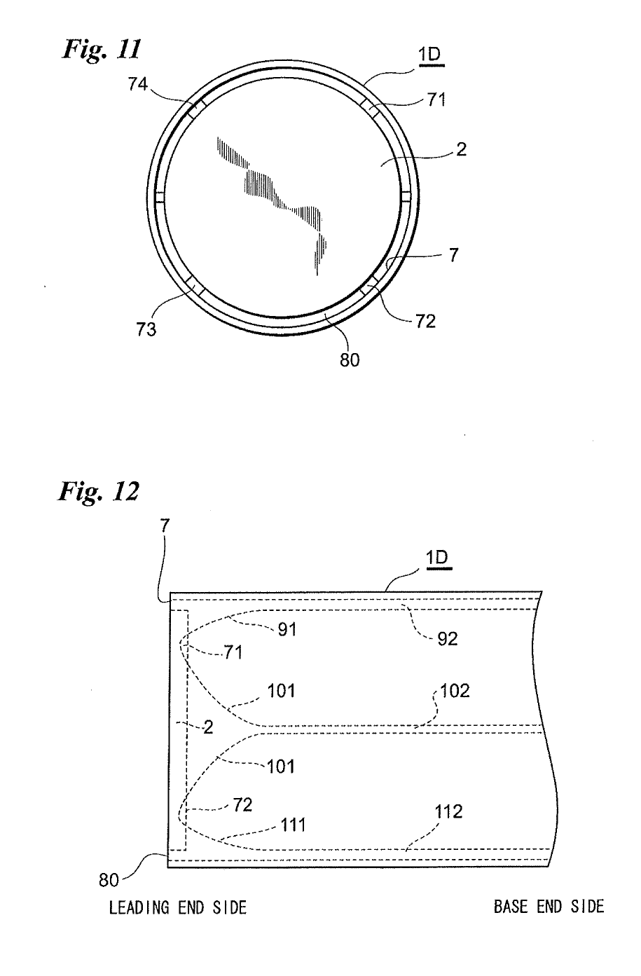

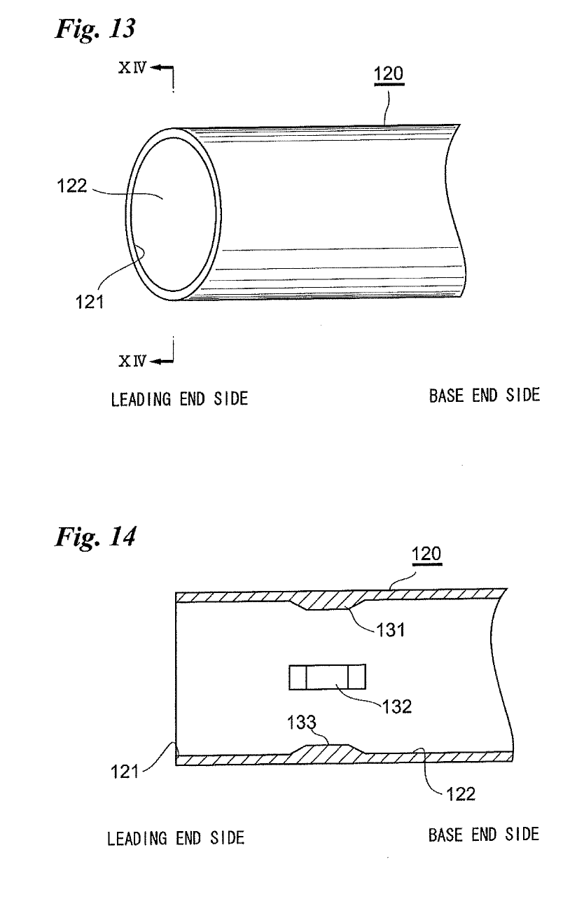

FIGS. 13 to 18 show still another preferred embodiment utilizing an overtube 120.

FIG. 13 is a perspective view of the overtube 120.

The overtube 120 has a circular tubular shape to cover an endoscope insertion portion. An opening 121 is formed on the leading end face of the overtube 120 through which the leading end face of the endoscope insertion portion is exposed.

FIG. 14 is a partial cross-sectional view taken along the line XIV-XIV of FIG. 13.

The overtube 120 is formed with an insertion passage 122 for covering the endoscope insertion portion inserted therethrough. Protrusions 131, 132, and 133 are formed in a manner protruding inward in upper, lower, left, and right regions on the insertion passage 122 a little bit nearer the base end than the leading end face. These protrusions 131, 132, and 133 are used to position the endoscope insertion portion within the overtube 120, and also have the same height.

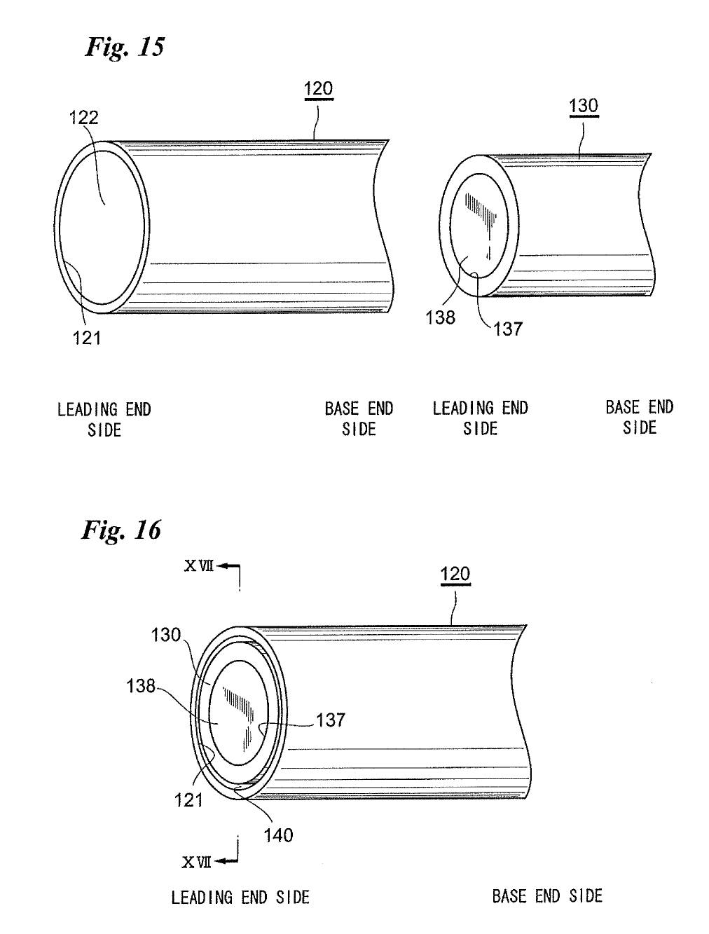

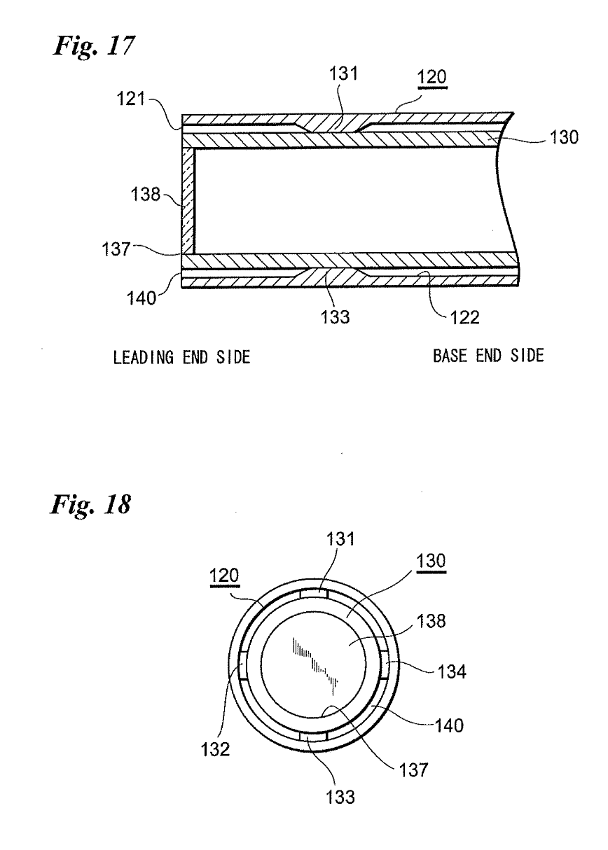

FIG. 15 is a perspective view of the overtube 120 and the endoscope insertion portion 130, FIG. 16 is a perspective view of the endoscope insertion portion 130 covered with the overtube 120, FIG. 17 is a cross-sectional view taken along the line XVII-XVII of FIG. 16, and FIG. 18 is a front view of the endoscope insertion portion 130 covered with the overtube 120.

Referring to FIG. 15, a glass plate 138 is fixed to the leading end face of the endoscope insertion portion 130. When the endoscope insertion portion 130 is inserted through the insertion passage 122 of the overtube 120, the glass plate 138, which is fixed to the observation window 137 on the leading end face of the endoscope insertion portion 130, is exposed through the opening of the overtube 120 as shown in FIG. 16. Since the protrusions 131, 132, 133, and 134 are formed in a manner protruding inward on the insertion passage 122 of the overtube 120, an opening portion 140 is formed, as shown in FIGS. 17 and 18, in a manner opened all around the endoscope insertion portion 130 in front view. A gas-feed passage 122 is also formed between the overtube 120 and the endoscope insertion portion 130 through which gas fed from the gas-feed device 10 flows. The gas flowing through the gas-feed passage 122 is ejected through the opening portion 140 in a manner surrounding the glass plate 138. Similarly, as mentioned above, gas streams generated by the gas ejected through the opening portion 140 blocks gas streams flowing toward the glass plate 138. This prevents floating matters from attaching to the glass plate 138.

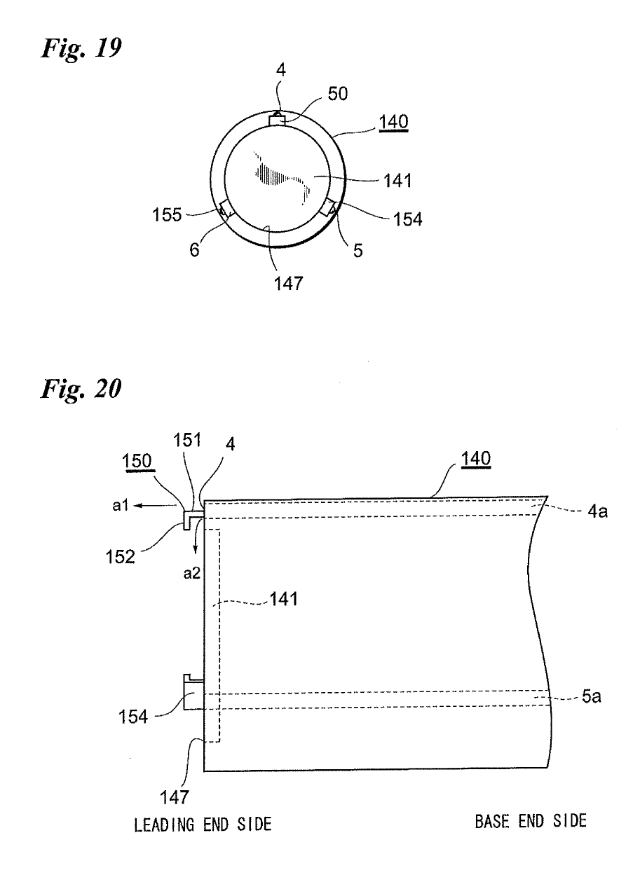

FIGS. 19 and 20 show a further preferred embodiment. FIG. 19 is a front view of an endoscope insertion portion 140, and FIG. 20 is a side view of the endoscope insertion portion 140. In these figures, components identical to those shown in FIGS. 1 to 4 are designated by the same reference numerals to omit the descriptions thereof.

A glass plate 141 is fixed to the observation window 147 on the leading end face of the endoscope insertion portion 140. Gas-feed ports 4, 5, and 6 are formed around the glass plate 141. Deflection members 150, 154, and 155 for partially deflecting the flow of air ejected through the gas-feed ports 4, 5, and 6 are installed over the gas-feed ports 4, 5, and 6.

Referring to FIG. 20, the deflection member 150 includes a support member 151 provided in a standing manner in the direction of gas feed through the gas-feed port 4 (leftward in FIG. 20) and a bent member 152 formed by bending an upper part of the support member 151 toward the glass plate 141.

Gas ejected through the gas-feed port 4 partially flows forward from the endoscope insertion portion 140 as indicated by the arrow a1. This removes floating matters from sight. The gas ejected through the gas-feed port is also guided partially to the surface of the glass plate 141 along the bent member 152 as indicated by the arrow a2. The gas guided to the surface of the glass plate 141 removes floating matters as they attach to the surface of the glass plate 141. The same applies not only to the deflection member 150 provided over the gas-feed port 4 but also to the other deflection members 154 and 155.

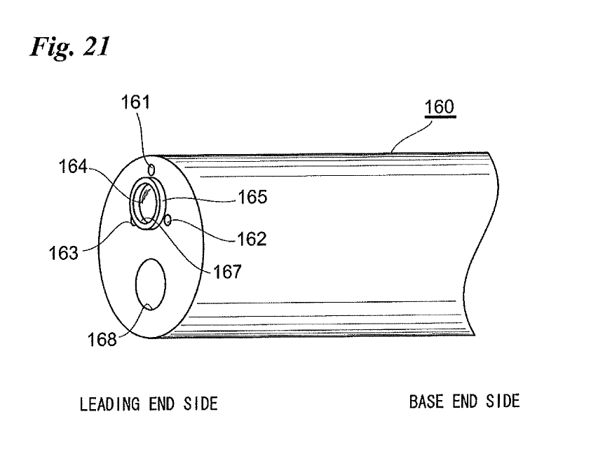

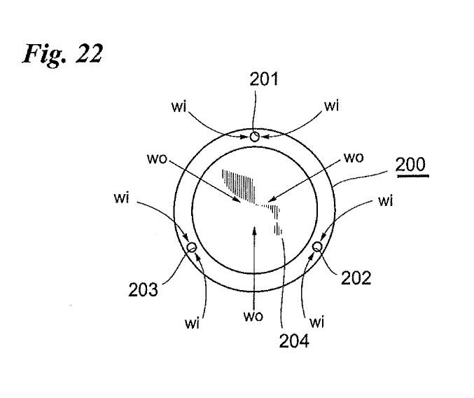

FIG. 21 is a perspective view of an endoscope insertion portion 160 of a flexible endoscope according to a still further preferred embodiment.

An imaging lens 164 is attached to the observation window 167 on the leading end face of the insertion portion 160 of the flexible endoscope. Around the imaging lens 164, a protective barrier 165 is provided in a manner standing and surrounding the imaging lens 164. Gas-feed ports 161, 162, and 163 are formed on the outside of the protective barrier 165. A forceps channel 168 is also formed in the leading end face of the endoscope insertion portion 160.

Gas feed through the gas-feed ports 161, 162, and 163, which removes floating matters in front of the imaging lens 164 as mentioned above, may cause gas streams and floating matters caught in the gas streams may attach to the surface of the imaging lens 164. However, the protective barrier 165, which is formed around the imaging lens 164, can prevent the gas streams from flowing onto the surface of the imaging lens 164 and thereby the floating matters from attaching to the surface of the imaging lens 164 as mentioned above.

Although the protective barrier 165 shown in FIG. 21 entirely surrounds the imaging lens 164, multiple protective barriers may be provided separately in a standing manner as shown in FIG. 7 or 8, or an opening may be formed in a manner surrounding the imaging lens 164 as shown in FIG. 11. Alternatively, an overtube may be utilized to form an opening surrounding the endoscope insertion portion 160 as shown in FIG. 16, or deflection members may be provided over the gas-feed ports 161, 162, and 163 for partially ejecting gas forward and partially guiding gas to the imaging lens 164 as shown in FIG. 19.

What is claimed is:

1. An endoscopic device comprising:
an endoscope insertion portion having a leading end portion, a base end portion, and a longitudinal shaft;
an observation optical member provided in the leading end portion of the endoscope insertion portion and having a surface exposed outward;
a convex portion formed around the observation optical member; and
a gas-feed port formed on the convex portion or on the outside of the convex portion with respect to the observation optical member and configured to eject gas therethrough in the direction of observation from the observation optical member,
wherein the convex portion is more protrudent than the surface of the observation optical member in the direction of observation.

2. The endoscopic device according to claim 1, wherein
the gas-feed port is formed at a position farther from the center of the convex portion or the observation optical member than the convex portion.

3. The endoscopic device according to claim 1, wherein
the convex portion is a protective barrier provided in a standing manner around the observation optical member.

4. The endoscopic device according to claim 3, wherein
at least one of the outer and inner walls of the protective barrier is inclined in a manner spreading downward.

5. The endoscopic device according to claim 3, wherein
the protective barrier is provided between the observation optical member and the gas-feed port in a manner surrounding the observation optical member.

6. The endoscopic device according to claim 3, wherein
the protective barrier is provided between the observation optical member and the gas-feed port only at a position facing the gas-feed port.

7. The endoscopic device according to claim 3, wherein
the protective barrier is not provided between the observation optical member and the gas-feed port but provided at a position opposed to the gas-feed port with respect to the observation optical member.

8. The endoscopic device according to claim 7, wherein
the outer wall of the protective barrier is inclined in a manner spreading downward.

9. The endoscopic device according to claim 1, being a rigid endoscopic device or a flexible endoscopic device.

10. The endoscopic device according to claim 1, further comprising a gas-feed device for feeding the gas-feed port with gas to be ejected through the gas-feed port.
